(19) United States
(12) Patent Application Publication
(10) Pub. No.: US 2009/0196833 A1
(43) Pub. Date: Aug. 6, 2009

(54) COMPOSITIONS COMPRISING TOPICAL DPD INHIBITORS AND METHODS OF USING SAME IN THE TREATMENT OF HAND-FOOT SYNDROME

(75) Inventors: William Paul Peters, Fernandina Beach, FL (US); Mukur Gupta, Morrisville, NC (US)

Correspondence Address: SEED INTELLECTUAL PROPERTY LAW GROUP PLLC, 701 FIFTH AVE, SUITE 5400, SEATTLE, WA, 98104, US

(73) Assignee: ADHEREX TECHNOLOGIES INC., Ottawa (CA)

(21) Appl. No.: 12/367,367

(22) Filed: Feb. 6, 2009

Related U.S. Application Data
(60) Provisional application No. 61/026,624, filed on Feb. 6, 2008.

(51) Int. Cl. A61K 9/12; A61K 31/513

(52) U.S. Cl. 424/45; 514/274

(57) ABSTRACT

Topical formulations comprising inhibitors of dihydropyrimidine dehydrogenase (DPD), thymidine phosphorylase (TP) and/or uridine phosphorylase (UP) enzyme inhibitors are provided for the treatment of hand-foot syndrome (HFS) in cancer patients undergoing treatment with 5-FU and 5-FU prodrugs.

### BACKGROUND

1. Technical Field

This invention relates generally to cancer therapy, and more particularly to topical formulations comprising inhibitors of dihydropyrimidine dehydrogenase (DPD), thymidine phosphorylase (TP) and/or uridine phosphorylase (UP), and methods of using such formulations in reducing the frequency and/or severity of hand-foot syndrome caused by 5-FU and/or 5-FU prodrugs.

2. Description of the Related Art

Hand-foot syndrome (HFS) is a well described, cumulative, dose limiting toxicity of certain commonly utilized cancer chemotherapy agents, particularly the fluroropyrimidines. Symptoms typically occur within the first few cycles of therapy and initially include numbness and tingling in the hands and feet. This is followed by plamar and plantar erythema, with subsequent blistering. The nature of this toxicity results in substantial patient discomfort and delays in treatment. Drugs most frequently implicated in causing HFS include 5-fluorouracil, liposomal doxorubicin (Doxil), cytarabine, docetaxel and 5-FU prodrugs, such as capecitabine (Xeloda®), and tegafur.

5-Fluorouracil (5-FU) has been clinically used to treat solid tumors in cancer patients for over three decades (Ansfield et al., Cancer 39: 34-40, 1977; Grem et al., Cancer Treat Rep 71: 1249-1264, 1987; Chabner et al., Cancer, Principles and Practice of Oncology, 2^{nd} Ed, pp 287-328 Philadelphia, Pa.: J B Lippincott Co, 1985). 5-FU must be activated by metabolic conversion to fraudulent uridine nucleotides (e.g., FUMP, FUDP, FUTP) and fraudulent deoxyuridine nucleotides (e.g., FdUMP, FdUDP, FdUTP) that interfere with DNA synthesis and RNA functions (reviewed in Meyers, Pharmacol Rev, 33: 1-15, 1981; Dasher et al., Pharmac Ther 48: 189-222, 1990). Because 5-FU differs from uracil, its natural counterpart, by only a fluorine substitution in the 5-position, it is readily activated in cancer patients. Unfortunately, its structural similarity to uracil also accounts for its rapid and extensive conversion to breakdown products that have no antitumor activity. This metabolic process is referred to as inactivation. 5-FU is rapidly inactivated by the enzyme dihydropyrimidine dehydrogenase (DPD: EC 1312, uracil reductase) (Meyers, Pharmacol Rev, 33: 1-15, 1981; Dasher et al., Pharmac Ther 48: 189-222, 1990). Therefore, the antitumor efficacy of 5-FU for treating cancer relies on the delicate balance between metabolic conversion to antitumor nucleotides (activation) and metabolic conversion to useless metabolites (inactivation).

Capecitabine (Xeloda®), a 5-FU prodrug, is approved and widely used for treatment of patients with breast cancer and colon cancer. However, as with 5-FU, capecitabine usage is associated with frequent HFS at the labeled dose and frequently require modification of the dosage for continued use. The reported incidence of HFS with capecitabine is approximately 60% for all grades and 17% for high grade (Xeloda® Prescribing Information, April 2006).

With capecitabine, multiple metabolic conversions are required for its therapeutic and toxic effects. The cause of HFS following treatment with 5-FU and 5-FU prodrugs treatment appears to result from catabolic products of 5-FU, most likely FBAL, which are produced by the metabolism of 5-FU in skin tissues. However, some data also suggest that capecitabine induced HFS results from excess activation of capecitabine in keratinocytes via thymidine phosphorylase (Fischel 2004). Thus, inhibition of the conversion of the 5-FU prodrug to 5-FU via inhibition of thymidine phosphorylase (TP) or uridine phosphorylase (UP), or catabolism of the 5-FU via inhibition of DPD, offer potential ways of interrupting the development of HFS.

The mechanism of HFS is unclear and despite trials using various topical agents, there is currently no established preventative or therapeutic strategy for effectively addressing this condition (Gressett 2006). The typical approach to a patient who has experienced HFS is to wait for the symptoms to resolve to grade 1 and then reduce the dose of the suspected chemotherapy drug for subsequent cycles.

Eniluracil is an irreversible inhibitor of dihydropyrimidine dehydrogenase (DPD) which modulates the metabolism of 5-FU (and endogenous uracil) by inhibiting the DPD mediated breakdown of 5-FU. A number of studies have demonstrated this ability of eniluracil to greatly enhance the bioavailability of orally administered 5-FU (Grem 2000; Keith 2002; Guo 2003). Studies using systemically administered DPD inhibitors in combination with 5-FU, in an effort to improve the bioavailability and efficacy of 5-FU treatment, revealed that the frequency of HFS syndrome was lower when these treatments were combined (Hoff 1995; Smith 2000; Rothenberg 2002). In these studies, HFS, of any grade, occurred in under 5% of all treated patients, consistent with a blockade of 5-FU catabolism. However, phase III studies failed to establish a therapeutic benefit of combined eniluracil and 5-FU and development was terminated.

At the time, the reasons for clinical failure of the combination therapy were not understood, however subsequent evidence suggests that at the dose and schedule employed, eniluracil was also acting as a competitive inhibitor of anabolism of 5-FU, thereby counteracting the intended benefits of the combination therapy. Indeed, recent evidence suggests that eniluracil can also function as a reversible inhibitor of thymidine phosphorylase (TP) and uridine phosphorylase (UP) in human tissue (Fourie et al. 2007). Thus, at selected concentrations, eniluracil is capable of inhibiting both the catabolism of 5-FU and 5-FU prodrugs by irreversibly inhibiting DPD and partially inhibiting the anabolism of 5-FU and 5-FU prodrugs by reversible inhibition of TP and UP. The inhibition of one or a combination of these three enzymes by eniluracil, or other inhibitors, could thus interrupt the production of the products needed for causation of HFS.

There remains an important and unmet need in the art for identifying optimal formulations and administration approaches for DPD inhibitors used in combination with 5-FU and 5-FU prodrugs in order to reduce the frequency and/or severity of HFS. The present invention fulfils these needs and offers other related advantages.

### BRIEF SUMMARY

It has been found that locally administered formulations comprising DPD, TP and/or UP inhibitors can effectively inhibit activity of these enzymes in the skin of animals, without significant effects on systemic 5-FU pharmacokinetics or systemic enzyme activity. As a result, the present invention provides topical formulations and methods for reducing the frequency and/or severity of HFS by proper dosing and administration DPD, TP and/or UP inhibitors locally to the hands and/or feet of a patient undergoing treatment with 5-FU or 5-FU prodrug.

Therefore, according to one aspect of the present invention, there are provided methods for reducing the frequency and/or severity of Hand-Foot Syndrome (HFS) in a patient undergoing treatment with 5-FU or a 5-FU prodrug, the methods comprising contacting the hands and/or feet of the patient with a topical formulation comprising an effective dose of a DPD TP and/or UP inhibitor. As demonstrated herein, such formulations can effectively inhibit DPD activity in the skin, e.g, in the hands and/or feet of a patient, without inhibiting systemic DPD activity in the patient and without effecting systemic 5-FU metabolism.

In one preferred embodiment, the topical formulation comprises an irreversible DPD inhibitor.

In another aspect, the DPD inhibitor used in the topical formulation may also be a TP and/or UP inhibitor.

In yet another aspect, a topical formulation of the invention may comprise a TP and/or UP inhibitor, separate or in combination with a DPD inhibitor.

The topical formulation can be in any suitable or conventional form, illustrative examples of which include an ointment, cream, lotion, aerosol spray, roll-on liquid, pad form, and the like. In certain embodiments, additional compounds are added which restrict blood flow to the area or by other means reduce systemic absorption of the DPD inhibitor.

The concentration of DPD inhibitor present in a topical formulation of the invention can be any concentration effective to achieve the desired local DPD inhibition while not substantially effecting systemic DPD activity. In certain embodiments, the concentration of DPD inhibitor in a topical formulation will range from about 0.001 to about 0.05 w/w.

Following application, the topical formulation may be optionally removed after a sufficient exposure time has elapsed. For example, in certain embodiments, the topical formulation will be substantially removed or washed from the skin after an exposure time of about 1 to about 60 minutes. In other embodiments, the topical formulation is substantially removed or washed from the skin after an exposure time of about 1 to 20 or 1 to 10 or 1 to 5 or 1 to 3 minutes.

The topical formulation may be applied before, at the same time as, or after 5-FU or 5-FU prodrug treatment, and the topical formulation may be applied one or multiple times during each courses of 5-FU or 5-FU prodrug treatment. In certain embodiments, the topical formulation is applied prior to 5-FU or 5-FU prodrug treatment, for example about 5 min to 72 hours prior to administration of 5-FU or 5-FU prodrug to a patient.

According to another aspect of the invention, there are provided topical formulations for reducing the frequency and/or severity of Hand-Foot Syndrome (HFS) in a patient undergoing treatment with 5-FU or a 5-FU prodrug, the topical formulation comprising an effective dose of an irreversible DPD inhibitor. Preferably, the effective dose of DPD inhibitor in the topical formulation inhibits DPD activity in the hands and/or feet but does not result in systemic DPD inhibition and does not effect 5-FU or 5-FU prodrug pharmacokinetics. In a particular embodiment, the concentration of DPD inhibitor in the topical formulation is about 0.001 to about 0.08 w/w. The topical formulation may be in any suitable or convenient form, for example selected from the group consisting of an ointment, cream, lotion, aerosol spray, roll-on liquid and pad form, as further described herein.

### BRIEF DESCRIPTION OF THE DRAWINGS

FIG. 1 shows a plasma concentration time profile for 5-FU following in vivo administration.

FIG. 2 shows a plasma concentration time profile for 5-FU following administration of capecitabine in vivo.

FIG. 3 shows that a single administration of an illustrative topical formulation comprising eniluracil had no effect on systemic 5-FU pharmacokinetics in vivo.

FIGS. 4A-D show the pharmacokinetic profile of individual formulations administered in vivo on days 0, 7 and 14.

FIG. 5 shows the extent and timing of DPD inhibition following application of topical eniluracil formulations.

### DETAILED DESCRIPTION

As noted above, the present invention is based in part on the discovery that topical formulations comprising DPD, TP and/or UP inhibitors, when applied using proper dosing and exposure times, are capable of blocking DPD, TP and/or UP enzymatic activity in the skin of animals, without significant effects on systemic DPD, TP and/or UP enzyme activity or 5-FU pharmacokinetics. Accordingly, the topical formulations and methods of the invention may be used in the treatment of hand-foot syndrome (HFS) by blocking the catabolism and anabolism of 5-FU and 5-FU prodrug locally in the skin (e.g., the hands and/or feet) of a patient being treated.

Accordingly, the treatment methods of the invention generally comprise any application of a topical formulation comprising one or more DPD, TP and/or UP inhibitors that results in a measurable clinical benefit to a patient undergoing treatment with 5-FU or a 5-FU prodrug, generally in the form of preventing the development of HFS, or in reducing the frequency and/or severity of HFS.

In certain preferred embodiments, a DPD inhibitor used in the topical formulations and methods of the present invention is an irreversible inhibitor of the DPD enzyme, such as eniluracil. In some cases, the inhibitor of the DPD enzyme may be a reversible inhibitor of DPD, such as CDHP. In certain other embodiments, the DPD inhibitor is an inhibitor of the DPD enzyme that is also an inhibitor of the TP and/or UP enzymes. In other embodiments, the topical formulation comprises a TP and/or UP inhibitor, alone or in combination with a DPD inhibitor.

In more particular embodiments, the DPD inhibitor may include, but is not limited to, a DPD inhibitor comprising a 5-substituted uracil compound, or a prodrug thereof, particularly a uracil compound substituted in the 5-position by a halogen atom, a C₂₋₄ alkenyl group (e.g., vinyl) optionally substituted by halogen (e.g,. 2-bromovinyl, 1-chlorovinyl or 2-bromo-1-chlorovinyl), a C₂₋₆ alkynyl group optionally substituted by a halogen atom, a cyano group, or a C₁₋₄ alkyl group substituted by halogen (e.g., trifluoromethyl).

In other embodiments of the invention, the DPD inhibitor is selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil, 5-hex-1-ynyluracil, 5-vinyluracil, 5-trifluorouracil, 5-bromouracil, and 5-(2-bromo-1-chlorovinyl)uracil, or a prodrug thereof.

In still other embodiments, the DPD inhibitor is a prodrug of 5-bromovinyluracil, one illustrative compound being represented by the compound 1-β-D-arabinofuranosyl-(E)-5-(2-bromovinyl)uracil (also referred to as BV-araU or sorivudine). Certain illustrative prodrug compounds in this regard are described, for example, in U.S. Pat. No. 4,386,076, the disclosure of which is incorporated herein by reference.

In other particular embodiments of the invention, the DPD inhibitor is eniluracil or a prodrug of eniluracil, such as 5-ethynyl-2(1H)-pyrimidinone (eniluracil missing the 4-oxygen) (Porter, et al., Biochem. Pharmacol 47: 1165-1171, 1994), a nucleoside or deoxynucleoside derivative of eniluracil, a compound that is converted to eniluracil in vivo, and/or a derivative of a DPD inactivator that is converted to the inactivator in vivo. By way of example, such compounds can include nucleoside derivatives which contain a nucleobase corresponding to the above 5-substituted uracil compounds, for example nucleoside derivatives containing a ribose, 2′-deoxyribose, 2′,3′-dideoxyribose, arabinose or other cleavable sugar portion, which may additionally contain a 2′- or 3 ′-substituent such as a halogen or a 5′ substituent such as an ester. More particular examples of such nucleoside derivatives include 1-(B-D-arabinofuranosyl)-5-prop-1-ynyluracil and 2′,3′-dideoxy-5-ethynyl-3′-fluorouridine.

The specific dose of DPD, TP and/or UP inhibitor(s) present in a topical formulation of the invention, and optimal exposure time, may, of course, vary depending upon the particular inhibitor(s) used as well as the particular components employed in the formulation, however these are readily determinable by a skilled artisan in view of the present disclosure. If a topical formulation is effective for the local inhibition of DPD, TP and/or UP activity in the skin of an animal while not substantially effecting systemic enzyme activity, then such formulation is considered within the spirit and scope of the present invention.

Therefore, an “effective dose” of a DPD inhibitor, for example, is a dose effective for inhibiting DPD activity in the skin (e.g., hands and/or feet) of a patient without effecting substantial systemic DPD inhibition (e.g., systemic DPD inhibition is less than about 40%, 10%, 5% or 1% of the normal DPD activity in the patient). This definition of “effective dose” is similarly and correspondingly applicable to TP and/or UP inhibitors. Of course, an “effective dose” will also preferably be one that prevents the development of HFS, reduces the frequency of HFS and/or reduces the severity of HFS.

In certain embodiments, the concentration of DPD, TP and/or UP inhibitor(s) present in a topical formulation of the invention may be from about 0.0001 to about 0.5 w/w. In a more particular embodiment, the dose in the topical formulation is from about 0.0001 to about 0.05 w/w. In a more particular embodiment, the dose in the topical formulation is from about 0.0001 to about 0.01 w/w. In a more particular embodiment, the dose in the topical formulation is from about 0.001 to about 0.05 w/w. In another more particular embodiment, the dose in the topical formulation is from about 0.001 to about 0.01 w/w.

In related embodiments, the total dose of inhibitor (e.g., eniluracil) applied to a patient per application is in the range from about 0.001 to 5, 0.001 to 1 or 0.01-0.5 mg per application.

The topical formulation will generally be applied bilaterally to hands (e.g., from the wrists down) and feet (e.g., from the ankles down). The amount of the topical formulation applied to the patient can, of course, vary depending on the concentration of the inhibitor in the formulation and the desired target dose to be applied. In certain embodiments, the amount of topical formulation applied to a patient per application is in the range from about 1 to 20, 1 to 10, or 1 to 5 grams. Generally, it will be desired to concentrate the majority of the topical formulation on the palms of the hands and the soles of the feet. The topical formulation may be applied, massaged into and/or otherwise contacted with the skin for any suitable duration provided the exposure time is not such that systemic DPD, TP and/or UP activity is substantially inhibited. In certain embodiments, the duration of exposure time to the topical formulation is about 1-10 minutes, 1-5 minutes or 1-3 minutes per appendage, followed by removal or washing of any excess ointment off the hand and foot.

The topical formulation may be applied one or multiple times provided that the application allows for effective inhibition of skin DPD, TP and/or UP activity but does not result in substantial systemic DPD, TP and/or UP inhibition. For example, in certain embodiments, the topical formulation is administered once, twice, three times, four times, five times or more, as needed or desired, during each course of 5-FU or 5-FU prodrug treatment.

The topical formulations of the invention may comprise essentially any suitable components that are biologically compatible and that are effective for facilitating the local delivery of DPD, TP and/or UP inhibitor(s) in the skin, particularly the hands and feet, of a patient. Thus, the topical formulations may be in any convenient format, including ointments, creams, lotions, aerosol sprays, roll-on liquids, sticks, pad forms, etc., as long as local delivery of the inhibitor is achieved as described herein. Topical formulations may further comprise expedients which inhibit the systemic absorption of the DPD, TP and/or UP from the topical site(s). Examples include, for example, vasoconstrictors such as epinephrine which reduce or delay absorption of the inhibitor.

In certain embodiments, the topical formulations may be anhydrous or emulsions, such as oil and water emulsions. Whether anhydrous or emulsion type, formulations may further include any of a variety of pharmaceutically acceptable carriers, skin actives and/or other necessary or desired components.

Generally, suitable amounts of a given carrier may range, for example, from about 1 to about 99%, from about 5 to about 70%, from about 10 to about 40% by weight, etc. Illustrative carriers may include, but are not limited to, emollients, water, inorganic powders, foaming agents, emulsifiers, fatty alcohols, fatty acids, and the like, as well as combinations thereof.

Emollients include substances selected from, for example, polyols, esters and hydrocarbons.

Illustrative polyols include, for example, propylene glycol, dipropylene glycol, polypropylene glycol, polyethylene glycol, sorbitol, hydroxypropyl sorbitol, hexylene glycol, 1,3-butylene glycol, 1,2.6-hexanetriol, glycerin, ethoxylated glycerin, propoxylated glycerin, xylitol, and the like, as well as mixtures thereof Illustrative esters useful as emollients include, for example:

(1) Alkyl esters of fatty acids having about 10 to 20 carbon atoms. For example, methyl, isopropyl, and butyl esters of fatty acids may be used. Particular examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, cetyl lactate, and the like. In certain embodiments, C12-C15 alcohol benzoate esters are used.

(2) Alkenyl esters of fatty acids having about 10 to 20 carbon atoms, illustrative examples including oleyl myristate, oleyl stearate and oleyl oleate.

(3) Ether-esters such as fatty acids esters of ethoxylated fatty alcohols.

(4) Polyhydric alcohol esters, such as ethylene glycol mono and di-fatty acid esters, diethylene glycol mono- and di-fatty acid esters, polyethylene glycol (200-6000) mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

(5) Wax esters such as beeswax, spermaceti, myristyl myristate, stearyl stearate.

(6) Sterol esters, of which cholesterol fatty acid esters are examples thereof.

Illustrative hydrocarbons include, for example, mineral oil, polyalphaolefins, petrolatum, isoparaffin, polybutenes, and the like, as well as mixtures thereof.

Inorganic powders may also be used as carriers, alone or in conjunction with other carriers, examples of which include clays (such as Montmorillonite, Hectorite, Laponite and Bentonite), talc, mica, silica, alumina, zeolites, sodium sulfate, sodium bicarbonate, sodium carbonate, calcium sulphate, etc., and mixtures thereof.

Aerosol propellants may also be used as carriers. Propellants are normally based on volatile hydrocarbons such as propane, butane, isobutene, pentane, isopropane and mixtures thereof. Philipps Petroleum Company is a source of such propellants under trademarks including A31, A32, A51 and A70. Halocarbons including fluorocarbons and dimethyl ether represent other illustrative propellants.

Emulsifiers may constitute at least a portion of the carrier for compositions according to the present invention, illustrative examples of which include, nonionic, anionic, cationic, or amphoteric emulsifying agents. They will typically range in amounts anywhere from about 0.1 to about 20% by weight, however this may vary depending on the particular emulsifier and the context of its intended use. Illustrative nonionic emulsifiers include, for example, alkoxylated compounds based on C10-C22 fatty alcohols and acids and sorbitan. These materials are available, for instance, from the Shell Chemical Company under the Neodol trademark. Copolymers of polyoxypropylenepolyoxyethylene sold by the BASF Corporation under the Pluronic trademark are sometimes also useful. Alkyl polyglycosides available from the Henkel Corporation represent additional illustrative emulsifiers.

Illustrative anionic type emulsifiers include, for example, fatty acid soaps, sodium lauryl sulphate, sodium lauryl ether sulphate, alkyl benzene sulphonate, mono- and di-alkyl acid phosphates, sarcosinates, taurates and sodium fatty acyl isethionate.

Illustrative amphoteric emulsifiers include, for example, such materials as dialkylamine oxide and various types of betaines (such as cocamidopropyl betaine).

Preservatives such as methyl paraben and propyl paraben may also be used, as desired, for example to prevent microbial contamination.

In a specific embodiment of the invention, the topical formulation is a petroleum-based ointment (e.g., Aquaphor®: petrolatum, mineral oil, cresin, and lanolin alcohol) compounded, for example, using a base. For example, in one illustrative embodiment, DPD inhibitor (e.g., eniluracil powder) is first dissolved in a base solution (e.g., 0.15 M NaOH) followed by levigation to ensure the inhibitor is fully distributed throughout the ointment. Of course, it will be understood that the amount of the inhibitor and ointment added during compounding can be varied to give rise to the desired formulation strength.

Following application of a topical formulation of the invention, the level of DPD activity in a patient or tissue can be determined using conventional methodologies. The normal range for DPD enzyme activity in man has been established to be about 0.064-0.314 nmol/min/mg in PBMC by measurement of the enzyme activity in peripheral blood mononuclear cells (PBMC), as the latter have been shown to mimic liver DPD activity.(Lu 1993; Chazal, 1996; Bocci et al. Clinical Pharm & Therapeutics 2006,80(4) 384-95). Therefore, in certain embodiments, following application of a topical formulation of the invention, local DPD inhibition is achieved in the skin, however systemic DPD activity does not fall substantially (e.g, greater than 1%, 5% or 10%) below normal DPD activity levels.

As discussed above, the topical formulations and methods of the present invention are useful in treating or preventing HFS in patients undergoing chemotherapy with agents including 5-FU and/or 5-FU prodrugs. Many such 5-FU and 5-FU prodrugs are known. A prodrug of 5-FU is a compound which is metabolized in vivo to 5-fluorouracil and may include, by way of illustration, 5-fluorouridine, 5-fluorocytidine, 5-fluoro-2-deoxyuridine, 5-fluoro-2-deoxycytidine, 5-fluoroarabinosyluracil, and their 5′-esters, including phosphate esters. Other illustrative compounds include 5′-deoxy-4′,5-fluorouridine, 5′-deoxy-5-fluorouridine, 1-(2-tetrahydrofuranyl)-5-fluorouracil, a 1-C₁₋₈ alkylcarbamoyl-5-fluorouracil derivative, 1-(2-tetrahydrofuryl)-5-fluorouracil, Ftorafur (Tegafur, an oral 5-FU prodrug that is widely used in Asian countries), and 5′-deoxy-5-fluoro-N-[(pentyloxy)carbonyl]-cytidine (capecitabine, marketed by Roche Laboratories Inc. as Xeloda®), or a compound that is converted to 5-FU in vivo.

5-FU and/or 5-FU prodrugs will generally be administered by their conventional and/or preferred routes and schedules of administration. For example, capecitabine doses will typically be in the range of those recommended by the FDA/National Comprehensive Cancer Network (NCCN) i.e., 1000-1250 mg/m² PO twice daily. In addition, 5-FU may be administered in a variety of dosages and schedules, including single dosages of, e.g., 500 mg/m2 to 750 mg/m2 once up to continuous low dose (e.g., 1-5 mg/m2) intravenous infusions for 28 days or more.

Methods for making DPD inhibitors and 5-FU prodrugs described herein are well known and may be carried out using any suitable conventional methodologies. For example, certain DPD inhibitors referred to above may be prepared by the methods described in Heterocycl. Chem. 19(3) 463-4 (1982) for the preparation of 5-ethynyluracil; J.Chem. Soc. Perkin Trans. 1(16), 1665-70 (1981) for the preparation of 5-(2-bromovinyl)uracil, 5-bromoethynyluracil and 5-(2-bromo-l-chlorovinyl)uracil; Nucleic Acid Chemistry, Vol. 2, 927-30 (1978) for the preparation 5-cyano-uracil; Nucleic Acids Research, 1 (1) 105-7 (1974) for the preparation of 5-vinyluracil; Z. Chern 17(11) 415-16 (1977) for the preparation of 5-trifluoromethyluracil; Nucleic Acids Research 3 (10),2845 (1976) for the preparation of 5-(1-chlorovinyl)uracil. Certain other compounds of the invention can be prepared in accordance with processes described in European Patent Specification No. 356166 for the preparation of 3′-fluoro-2′,3 ′-dideoxy-5-alkynyluridine compounds, such as 2′,3′-dideoxy-5-ethynyl-3′-fluorouridine, and European Patent Specification No. 272065 for the preparation of 5-alkynyluracil arabinosides, such as 1-(b-D-arabinofuranosyl)-5-prop-1-ynyluracil.

The following examples will further illustrate certain embodiments of the invention.

### EXAMPLES

### Example 1

### In-Vitro Determination of Eniluracil Dermal Irritation

The EpiDerm™ skin model system (MatTek) was used to assess the potential dermal irritation of eniluracil alone or its formulation. This system consists of normal, human-derived epidermal keratinocytes (NHEK) which have been cultured to form a multilayered, highly differentiated model of the human epidermis. The model contains organized basal, spinous, granular, and comified layers analogous to those found in vivo, and exhibits in vivo-like morphological and growth characteristics which are uniform and highly reproducible.

The EpiDerm™ system is mitotically and metabolically active. Markers of mature epidermis-specific differentiation such as pro-filaggrin, the K1/K10 cytokeratin pair, involucrin, and type I epidermal transglutaminase have been localized in the model. The MTT (3-[4,5-dimethylthiazol-2-yl]-2,5-diphenyltetrazolium bromide) conversion assay, which measures the NAD(P)H-dependent microsomal enzyme reduction of MTT (and to a lesser extent, the succinate dehydrogenase reduction of MTT) to a blue formazan precipitate, was used to assess cellular metabolism after exposure to a test article for various exposure times. The duration of exposure resulting in a 50% decrease in MTT conversion in test article-treated EpiDerm™ cultures, relative to control cultures, was determined (ET₅₀).

Eniluracil was prepared and administered to the test system at the three final concentrations of 1000 μM, 500 μM, and 100 μM, and tested at three exposure times of 8, 20, and 24 hours. The DMSO solvent control was tested at exposure times of 8 and 24 hours. The negative control was tested at exposure times of 4 and 24 hours, and the positive control was tested at exposure times of 4 and 8 hours. The results of the trial, presented in Table 1, confirmed that the three doses of eniluracil were well-tolerated by the EpiDerm™ skin model at topical exposures of up to 24 hours. The ET₅₀ value for the positive control, 1% Triton®-X-100, fell within two standard deviations of the historical mean (4.17 to 6.96 hours), thereby meeting the acceptance criteria. Based upon the results of other chemicals tested in this assay system historically, ET₅₀ values of greater than 24 hours are suggestive of materials with low acute dermal irritation potential. Within the confines of this study, the test articles resulted in ET₅₀ values notably longer than that obtained from the positive control, 1% Triton®-X-100.

TABLE 1
ET₅₀ values of various concentrations of eniluracil tested
in the EpiDerm ™ skin model

| Compound | ET₅₀ | |
|---|---|---|
| Concentration | (hours) | pH |
| Eniluracil (1000 uM) | >24 | 5 |
| Eniluracil (500 uM) | >24 | 5 |
| Eniluracil (100 uM) | >24 | 5 |
| 1% Triton-X-100 | 6.09 | NA |

### Example 2

### Evaluation of Eniluracil Ointment in the Epiderm™ Skin Model

EpiDerm™ cultures were tested in duplicate with eniluracil ointment at four exposure times of 4, 8, 16, and 24 hours. Eniluracil ointment was prepared by dissolving appropriate amounts of eniluracil in a sodium hydroxide solution and then levigating it with Aquaphor to obtain 0.0005-0.1% w/w. Hydrochloric acid in an amount equivalent to the sodium hydroxide was added to neutralize the ointment. The exposure time control was also exposed in duplicate for 4 and 24 hours. Table 2 below summarizes the ET₅₀ results of the EpiDerm™ assay for the test articles and the positive control, using the negative control results to determine the relative viability. Additionally, for the test articles, eniluracil 0.1% (w/w) and eniluracil 0.0005% (w/w), percent of control values were calculated using the test article, placebo ointment, as the placebo or vehicle control. The ET₅₀ value for the positive control, 1% Triton®-X-100, fell within two standard deviations of the historical mean (4.17 to 6.96 hours), thereby meeting the acceptance criteria. Finally, none of the test articles were observed to directly reduce MTT in the absence of viable tissue. Test article residues persisted on some of the treated tissues following the rinsing process at all exposure times for the test article, placebo ointment, and at the 8, 16, and 24 hour exposure times for the test articles, eniluracil 0.1% (w/w) and eniluracil 0.0005% (w/w). The test article residues presumably prolonged the exposure times relative to the reported exposure times.

TABLE 2
ET₅₀ values of various concentrations of eniluracil
tested in the EpiDerm ™ skin model

| | ET₅₀ | |
|---|---|---|
| Compound Concentration | (hours) | pH |
| Placebo Ointment | >24 | 5 |
| Eniluracil 0.1% (w/w) | >24 | 5 |
| Eniluracil 0.0005% (w/w) | >24 | 5 |
| 1% Triton-X-100 | 4.95 | NA |

### Example 3

### Evaluation of Topical Eniluracil in Mice

To evaluate the effect of topical administration of eniluracil on DPD activity in the skin and in the liver, various studies were conducted in mice. The DPD activity in the skin of mice treated with placebo was determined to be 1.4 pmol/min/mg (mean of DPD activity of placebo in Table 3) of protein and in the liver it was determined to be 2426.66 pmol/min/mg of protein (mean of DPD activity of placebo in Table 4). Mice in the studies had an exposure time of one hour i.e., eniluracil ointment was applied for one hour and then removed, unless otherwise specified.

DPD activity was measured according to the following procedure. All tissue samples were homogenized in ice-cold buffer (35 mM KH₂PO₄ buffer 1.5 mM DTT, pH=8) in the presence of protease inhibitors, centrifuged for 1 hour at 100,000×g at 4° C., and the supernatant (cytosolic fraction) was collected for use as the enzyme source. The reaction mixture for determining DPD activity in tissue samples consisted of 35 mM KH₂PO₄ buffer (pH=8), 5 mM MgCl₂, 1 mM DTT, 100 μM NADPH, 20 μM [6-¹⁴C]-5-FU, and 80 μL cytosolic extract in a final volume of 160 μL. All incubations were initiated by the addition of cytosolic extract and were conducted at 37° C. in a shaking water bath. Enzymatic activity were terminated after 15 to 30 minutes by boiling x 3min and subsequent frozen at −80° C. Precipitated protein was removed by centrifugation, the supernatant was filtered and 5-FU catabolites [6-¹⁴C]-5-fluroureidopropionic acid and [6-¹⁴C]-dihydro-5-fluorouracil formed by DPD, were separate by reverse-phase HPLC (mobile phase: 5 mM tetrabutylammonium hydrogensulfate and 1.5 mM potassium phosphate [pH=7.6]), and quantified by a Flow Scintillation Analyzer connected on line with the HPLC.

### A. Determination of Dose Range for Topical Eniluracil:

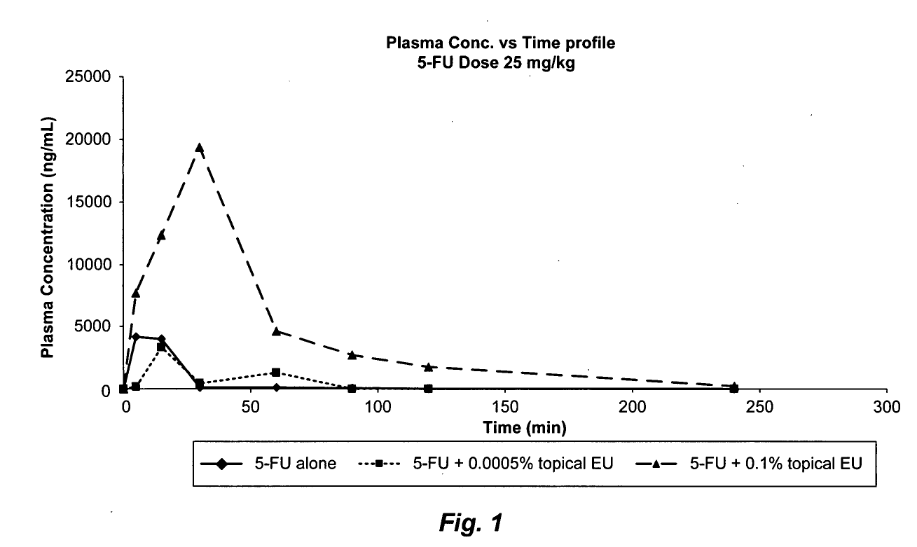

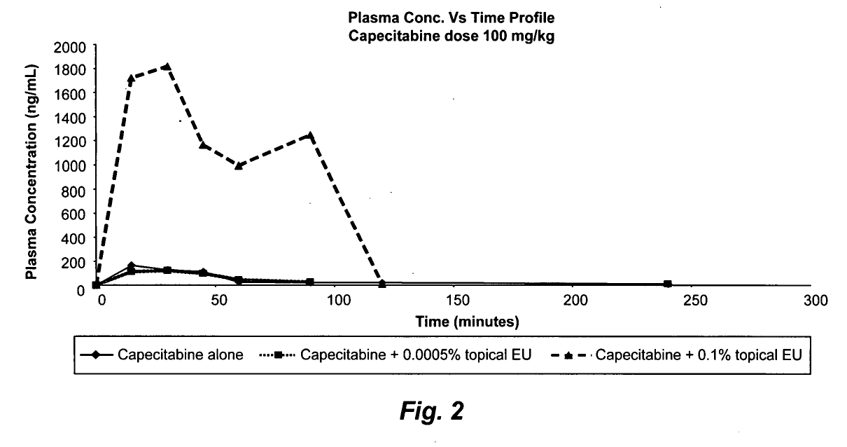

The dose range for topical administration of eniluracil in mice was determined by dosing 40-50 mg of placebo, 0.0005% w/w and 0.1% w/w of eniluracil ointment for one hour and then removing it with alcohol swab. Skin and liver samples were then collected at pre-determined time, and DPD activity was measured. Effects on skin DPD activity and liver DPD activity are listed in Table 3 and 4. These animals were also dosed with 25 mg/kg of 5-FU (administered after one hour of ointment application) to evaluate the effect of 0.1 % w/w and 0.0005% w/w on its pharmacokinetics. 5-FU plasma levels were determined. A plasma concentration time profile for 5-FU is shown in FIG. 1. A similar pharmacokinetic evaluation was also conducted with 100 mg/kg capecitabine (administered after one hour of ointment application). A plasma concentration time profile for 5-FU after administration of capecitabine is shown is FIG. 2. It is clear from the data that 0.0005% w/w had no effect on the skin and liver DPD activity, and it did not affect the pharmacokinetics of 5-FU (FIG. 1). On the other hand, 0.1% w/w totally inhibited DPD activity in skin and in liver, and it also affected the 5-FU pharmacokinetics. Similar effects were seen when the animals were dosed with capecitabine i.e., 0.0005% w/w did not affect the 5-FU pharmacokinetics after capecitabine administration but 0.1% w/w did affect the 5-FU pharmacokinetics after capecitabine administration (FIG. 2).

TABLE 3
DPD activity in the skin of the mice treated with (0.0005, and
0.1% (w/w)) of eniluracil ointment for 1 hour
Skin DPD activity (pmol/min/mg of protein)

| Time | | | 0.1% |
|---|---|---|---|
| Points | Placebo | 0.0005% (w/w) | (w/w) |
| 5 min | 1.74 | 1.41 | ND |
| 5 min | 1.70 | 1.30 | ND |
| 2 hrs | 1.18 | 1.29 | 0.33 |
| 2 hrs | 1.12 | 1.15 | 0 |
| 4 hrs | 1.53 | 1.64 | 0 |
| 4 hrs | 1.41 | 1.54 | 0 |

TABLE 4
DPD activity in the liver of the mice treated with
(0.0005, and 0.1% (w/w)) of eniluracil ointment for 1 hour
Liver DPD activity (pmol/min/mg of protein)

| Time | | | 0.1% |
|---|---|---|---|
| Points | Placebo | 0.0005% (w/w) | (w/w) |
| 5 min | 2339.53 | 2088.71 | 0 |
| 5 min | 2271.07 | 2227.77 | 0 |
| 2 hrs | 2516.14 | 1827.22 | 0 |
| 2 hrs | 2579.91 | 1770.27 | 0 |
| 4 hrs | ND | 1691.36 | 0 |
| 4 hrs | ND | 1598.79 | 92.14 |

### B. Effect of Various Doses of Eniluracil Ointment on Skin and Liver DPD Activity:

To evaluate the effect of various doses of topical eniluracil on skin and liver DPD activity, 40-50 mg of eniluracil ointment (0.001, 0.005, 0.01 and 0.05% w/w) was applied topically for one hour and then removed using an alcohol swab. Skin and liver samples were collected and DPD activity was measured. DPD activity was affected in both liver and skin (Table 5 and 6), except 0.001% w/w which affected DPD activity (33% inhibited) in skin only; DPD activity in liver remained unaffected. This demonstrates that in the mice where absorption of drug is rapid, 40-50 mg of 0.001% w/w is sufficient enough to block 33% of skin DPD activity without affecting the liver DPD activity.

TABLE 5
DPD activity and percent of DPD activity inhibited in
the liver of the mice treated with (0.001, 0.005,
0.01 and 0.05% (w/w)) topical eniluracil

| | | DPD Activity | Percent of DPD |
|---|---|---|---|
| Group | | (pmol/min/mg protein) | activity inhibited |
| 0.001% | w/w | 3009.14 | −24.00 |
| 0.001% | w/w | 2890.07 | −19.10 |
| 0.005% | w/w | 1625.52 | 33.01 |
| 0.005% | w/w | 1550.92 | 36.09 |
| 0.01% | w/w | 159.89 | 93.41 |
| 0.01% | w/w | 133.19 | 94.51 |
| 0.05% | w/w | 0 | 100.00 |
| 0.05% | w/w | 0 | 100.00 |

TABLE 6
DPD activity and percent of DPD activity inhibited
in the skin of the mice treated with
(0.001, 0.005, 0.01 and 0.05% (w/w)) topical eniluracil

| | | DPD Activity | Percent of DPD |
|---|---|---|---|
| Group | | (pmol/min/mg protein) | activity inhibited |
| 0.001% | w/w | 0.95 | 32.07 |
| 0.001% | w/w | 0.91 | 34.79 |
| 0.005% | w/w | 1.14 | 18.29 |
| 0.005% | w/w | 0.89 | 36.07 |
| 0.01% | w/w | 0.71 | 49.07 |
| 0.01% | w/w | 0 | 100.00 |
| 0.05% | w/w | 0 | 100.00 |
| 0.05% | w/w | 0 | 100.00 |

### C. Recovery of DPD Activity After 0.01% W/W Eniluracil Ointment:

To evaluate the recovery of DPD in the skin and in the liver, mice were treated with 0.01% w/w of eniluracil ointment (this dose was high enough to block significant amount of DPD activity in the skin and in the liver) for one hour and then the ointment was removed using an alcohol swab Skin and liver samples were collected and were analyzed for DPD activity. The results in Table 7 show that after 1 hour of application of 0.01% w/w of eniluracil ointment DPD activity was totally inhibited in skin and DPD activity in the liver was also significantly affected (Table 8). DPD activity in both the tissues returned to normal activity after day 6 for liver and day 4 for skin.

TABLE 7
DPD activity in the skin of the mice treated
with 0.01% w/w topical eniluracil

| | Mean DPD Activity | Percent of DPD |
|---|---|---|
| Time Points | (pmol/min/mg protein) | activity inhibited |
| Day 0 (1 hr after | 0.00 | 100.00 |
| application) | | |
| Day 2 | 1.34 | 4.29 |
| Day 4 | 1.62 | −15.71 |
| Day 6 | 2.75 | −96.43 |
| Day 8 | 3.04 | −117.14 |

TABLE 8
DPD activity in the liver of the mice treated with
0.01% w/w topical eniluracil

| | Mean DPD Activity | Percent of DPD |
|---|---|---|
| Time Points | (pmol/min/mg protein) | activity inhibited |
| Day 0 (1 hr after | 41.72 | 98.28 |
| application) | | |
| Day 2 | 1172.48 | 51.68 |
| Day 4 | 1157.53 | 52.30 |
| Day 6 | 2920.92 | −20.37 |
| Day 8 | 3175.38 | −30.85 |

### D. Effect on Skin and Liver DPD After a Short Exposure of 0.005% W/W Eniluracil Ointment:

In our earlier study, 0.005% w/w of eniluracil ointment was effective to block ˜33 % of DPD activity in the skin and 27% in the liver after 1 hour of application. In this study we evaluated the effect on DPD activity in the skin and the liver of mice treated with 0.005% (w/w) topical eniluracil for 2, 5, 15 and 30 minutes. Table 9 shows that DPD activity in the skin was affected even after a short exposure of topical eniluracil. Table 9 also shows that DPD activity in the liver was not affected after short exposure of topical eniluracil. The data is consistent with our previous study and also demonstrates that in mice a longer exposure is not needed to completely inhibit DPD activity in the skin.

TABLE 9
DPD activity and percent of DPD activity inhibited in
the skin and the liver of the mice treated with 0.005% w/w
of topical eniluracil for 2, 5, 15, and 30 minutes.

| | | Skin DPD | | | Percent |
|---|---|---|---|---|---|
| | | activity | | Liver DPD activity | of DPD |
| | | (pmol/min/mg | Percent | (pmol/min/mg | activity |
| Time | | protein) | inhibited | protein) | inhibited |
| 2 | min | 0 | 100.00 | 2657.72 | −9.52 |
| 2 | min | 0 | 100.00 | 2849.44 | −17.42 |
| 5 | min | 1.445 | −3.21 | 3248.27 | −33.86 |
| 5 | min | 1.481 | −5.79 | 3126.83 | −28.85 |
| 15 | min | 0.938 | 33.00 | 3154.15 | −29.98 |
| 15 | min | 0.496 | 64.57 | 3151.17 | −29.86 |
| 30 | min | 0 | 100.00 | 3138.76 | −29.34 |
| 30 | min | 0.78 | 44.29 | 2969.64 | −22.38 |

### Example 4

### Evaluation of Topical Eniluracil in Pigs

To evaluate the effect of single administration of topical eniluracil on skin DPD activity, pigs were dosed with 5 gms of 0.0005, 0.001, 0.005 and 0.01 % w/w for 30 minutes and then the ointment was wiped off using a Kimwipe®. Skin samples were collected at predetermined time points. To reduce the intra-pig variability, each pig was dosed with treatment on one site and placebo on the other. Table 10 shows the effect of individual doses on skin DPD activity at 0.5 hrs and 12 hours. (7 hours for the pig treated with 0.001% w/w). DPD activity was measured following the procedure noted above. It is clear from the data that all dose levels of eniluracil affected skin DPD activity and most of the DPD activity was recovered after 12 hours.

TABLE 10
Percent of DPD activity inhibited in the skin of the pigs
treated with 0.0005, 0.001, 0.005 and 0.01% w/w
of topical eniluracil for 30 minutes.

| | | 0.0005% w/w | 0.001% w/w | 0.005% w/w | 0.01% w/w |
|---|---|---|---|---|---|
| Time Points | | Percent of DPD activity inhibited | | | |
| 0.5 | hrs | 53.55 | 44.01 | 87.24 | 79.42 |
| 0.5 | hrs | 55.90 | 45.38 | 90.84 | 79.57 |
| 12 | hrs | 36.72 | 61.11* | 16.55 | −22.52 |
| 12 | hrs | 34.60 | 61.64* | 21.08 | −21.74 |

*7 hours not 12 hours

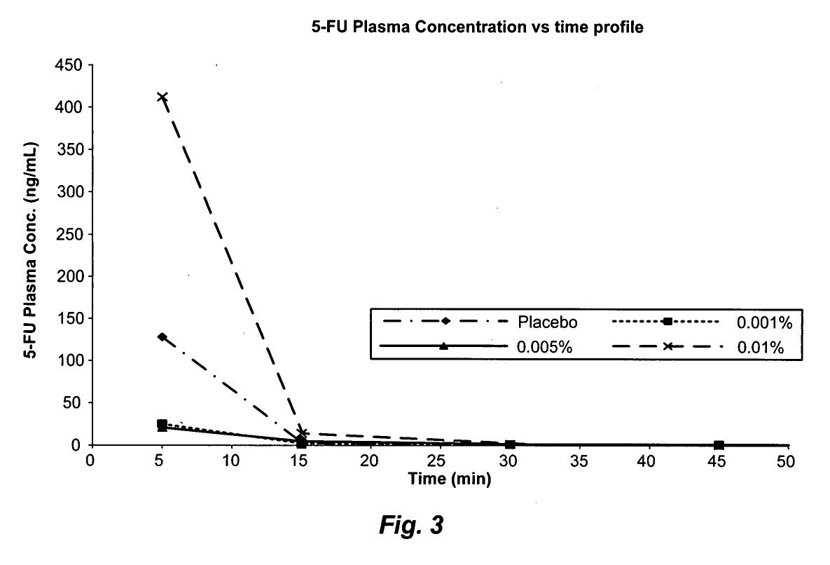

In another experiment, pigs were dosed with 0.001, 0.005 and 0.01% w/w of topical eniluracil for 30 minutes and then the ointment was wiped off using a Kimwipe®. These animals were also dosed with 0.5 mg/kg of 5-FU and blood and PBMC samples were collected at pre-defined time intervals. 5-FU plasma levels and DPD activities were determined. PBMC data in Table 11 shows that DPD activity was quite high in the treated animals compared to the placebo. Also, FIG. 3 shows that a single administration of topical eniluracil had no effect on 5-FU pharmacokinetics.

These data indicate that in pigs where absorption is slower compared to mice, DPD activity in the skin was inhibited whereas the systemic DPD activity remained unchanged. These animals continued to receive placebo, 0.001, 0.005 and 0.01% w/w of topical eniluracil, for 30 minutes and skin DPD activity was measured at Day 7 and 14. These animals were also dosed with 0.5 mg/kg of 5-FU on Day 7 and on Day 14.

TABLE 11
DPD activity in the PBMCs of the pigs treated with 0.001, 0.005
and 0.01% w/w of topical eniluracil for 30 minutes.

| | | Placebo | 0.001% (w/w) | 0.005% (w/w) | 0.01% (w/w) |
|---|---|---|---|---|---|
| 0 | hrs | 45.26 | 58.29 | 102.22 | ND |
| 0 | hrs | 29.81 | 57.21 | 62.60 | ND |
| 0.5 | hrs | 26.64 | 58.07 | 97.89 | 127.68 |
| 0.5 | hrs | 33.69 | 32.14 | 54.12 | 128.58 |
| 5 | hrs | 73.13 | 50.13 | 67.92 | 63.50 |
| 5 | hrs | 43.24 | 45.52 | 78.51 | 125.39 |

Following repeated administration of topical eniluracil (0.001, 0.005 and 0.01% w/w) for 30 min every day, for 6 days followed by a skin biopsy results, showed that on Day 7, i.e., after 24 hours there was no effect on the skin DPD activity; however, after 14 days of treatment with topical eniluracil (0.01% w/w 30 minutes every day) reduced DPD activity in the skin by approximately 15%. (Table 12).

TABLE 12
DPD activity in the pig skin treated with 0.001, 0.005 and
0.01% w/w of topical eniluracil for 30 minutes every day for 13 days
and sample was collected on day 14, 24 hours after administration.

| Time points | Placebo | 0.001% (w/w) | 0.005% (w/w) | 0.01% (w/w) |
|---|---|---|---|---|
| 7 days | 24.893 | 65.59 | 39.24 | 31.66 |
| 7 days | 25.006 | 73.99 | 39.55 | 33.93 |
| 14 days | 23.799 | 53.27 | 58.08 | 8.72 |
| 14 days | 28.695 | 53.40 | 77.01 | 5.83 |

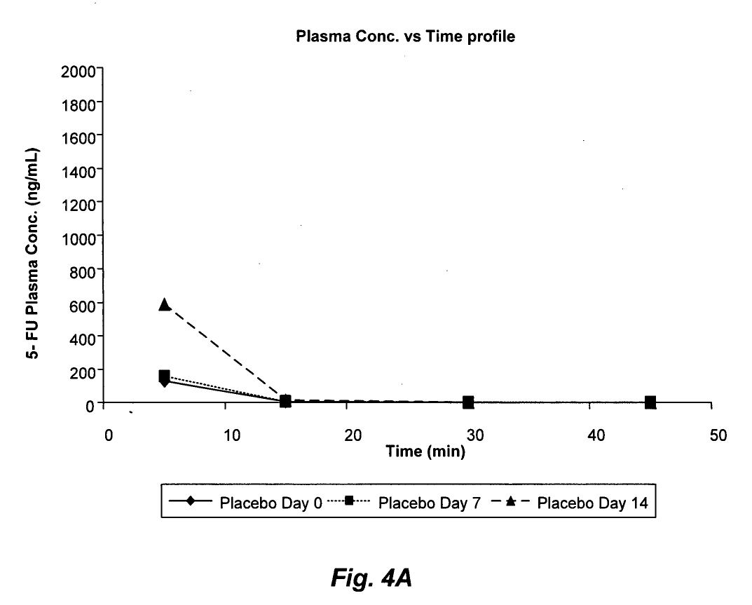

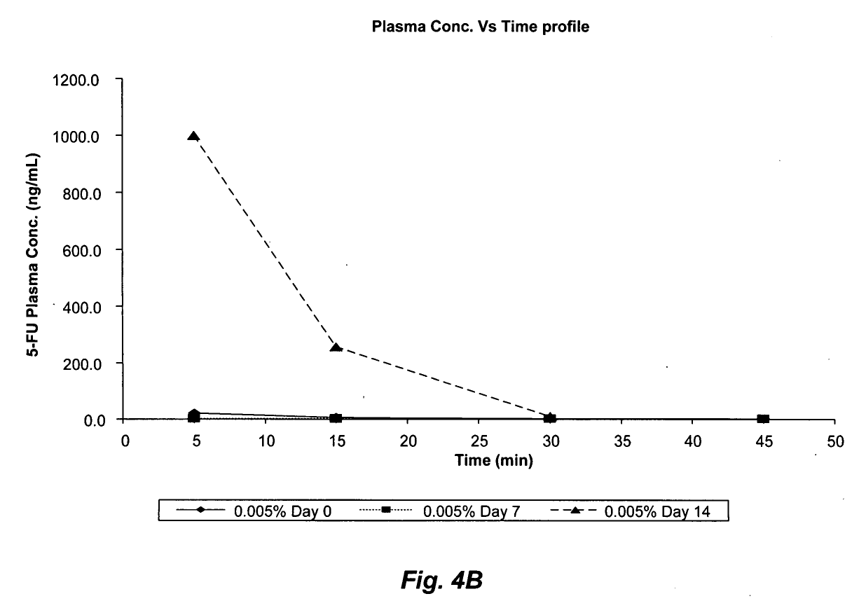

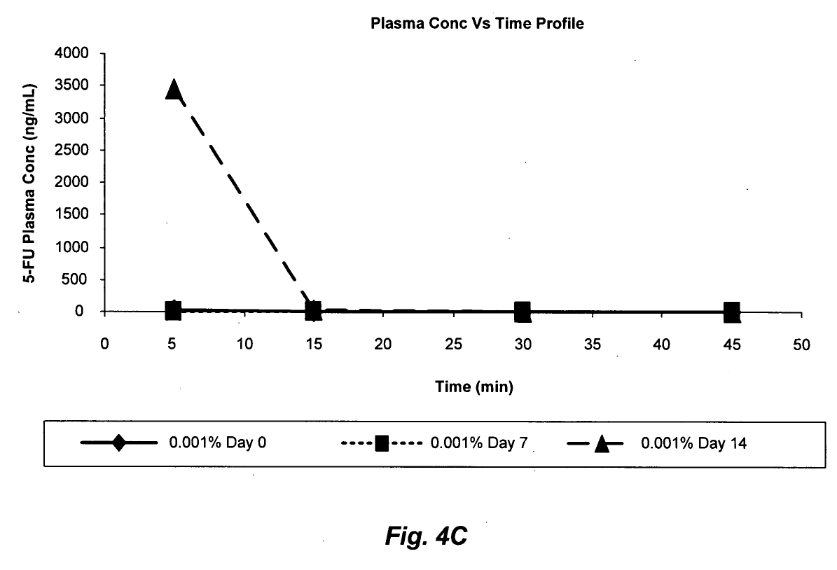

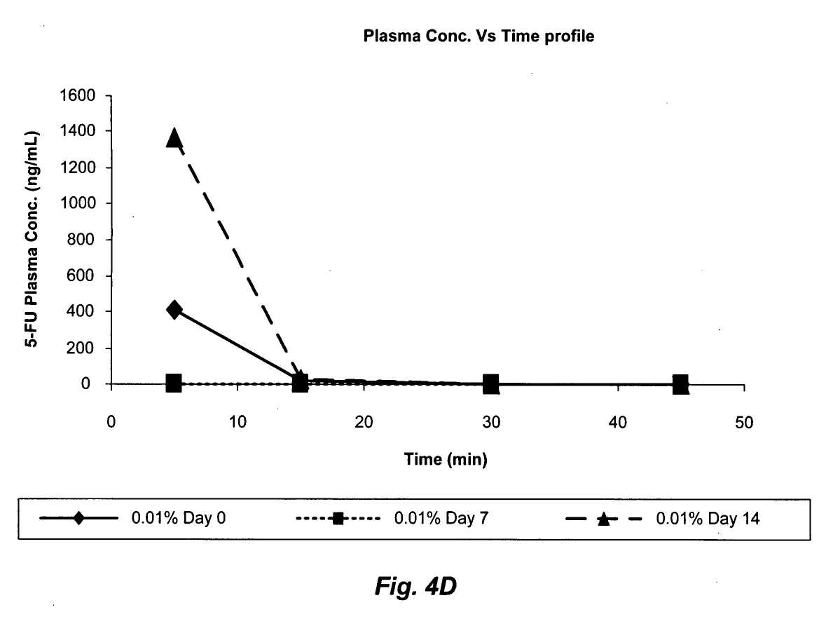

FIGS. 4A-D depict the pharmacokinetic profile of individual formulations on Day 0, Day 7 and Day 14. It is clear from the profiles that repeated application of topical eniluracil has no effect on 5-FU pharmacokinetics on day 7 but delays plasma 5-FU elimination at 14 days.

### Example 5

### Inhibitory Effects of Uridine and Thymidine on Thymidine Phosphorylase (TP)

Skin samples were suspended in 600 μl of homogenization buffer containing 20 mM potassium phosphate (pH 8.0), 1.5 mM dithiothreitol (DTT), and 5 ul/ml protease inhibitor (Sigma). Samples were homogenized and centrifuged at 36,000 rpm for one hour at 4° C. The supernatants were collected and protein concentration was measured using the Bradford assay (Biorad). Thymidine phosporylase (TP) enzyme activity was determined in an assay mixture containing 20 mM potassium phosphate (pH 8.0), 1 mM DTT, 1 mM EDTA, 2 μM 6-³H-Capecitabine (Moravek Biochemicals, Brea, Calif.), 200 μM unlabeled Capecitabine, varying concentrations of the inhibitor (100 μM, 10 μM, or 1 μM), and 80 μl of protein (6.0 mg/ml) in a final volume of 160 μl. Reactions were incubated at 37° C. for 30 minutes and then terminated by boiling for four minutes. To facilitate protein precipitation, the reactions were incubated at −80° C. for at least 20 minutes, thawed, and then centrifuged for 10 minutes at 14000 rpm at 4° C. The resulting supernatants were filtered through a 0.2 μm Acrodisc filter (VWR International, West Chester, Pa.) and samples were injected in duplicate (60 μl each) onto a HPLC (Hewlett-Packard 1050, Houston, Tex.) equipped with an automatic injector and on-line radioisotope flow detector (Radiomatic FLO-ONE Beta, Packard Instrument, Meriden, Conn.). All analyses were performed using a C18 reversed phase column (250×4.6 mm) (Alltech Associates, Inc, Deerfield, Ill.) and elution was carried out isocratically for 20 min at a flow rate of 1 ml/min with a mobile phase consisting of 1.5 mM potassium phosphate and 5.0 mM tetrabutylammonium hydrogen sulfate (pH 7.6) followed by 10 min with a flow rate of 1 ml/min with 80% acetonitrile for separation of 6-³H-Capecitabine (5′-deoxy-5-fluorouridine) from ³H-5-fluorouracil (product).

Based on these experiments, it was observed that both thymidine and uridine inhibit TP activity in a dose dependent fashion, with both compounds inhibiting TP activity by about 50% at a concentration of 100 uM. Thus, thymidine and uridine may be employed in the context of the compositions and methods of the invention where TP inhibition is desired.

### Example 6

### Effects of Eniluracil After Topical Administration of Skin DPD Activity, 5-FU Pharmacokinetics and Systemic DPD Activity In Vivo

This study was carried out to determine the plasma pharmacokinetics of eniluracil (EU) after topical administration and to evaluate the effect of topical eniluracil application on 5-FU pharmacokinetics, peripheral blood mononuclear cell (PBMC) dihydropyrimidine dehydrogenase (DPD) function, and levels of DPD activity in the skin in vivo. Eniluracil (0.005% and 0.01% w/w) formulated using Aquaphor® was applied topically (617 mg to a designated area) for 5 minutes every other day for 16 days starting on Day-2. 5-Fluorouracil was administered as a 6 mg/kg bolus injection on Day-3, Day 0 and Day 14. Samples were collected at predetermined time intervals to evaluate 5-fluorouracil pharmacokinetics, eniluracil pharmacokinetics, skin DPD activity and systemic DPD activity.

### Materials and Methods:

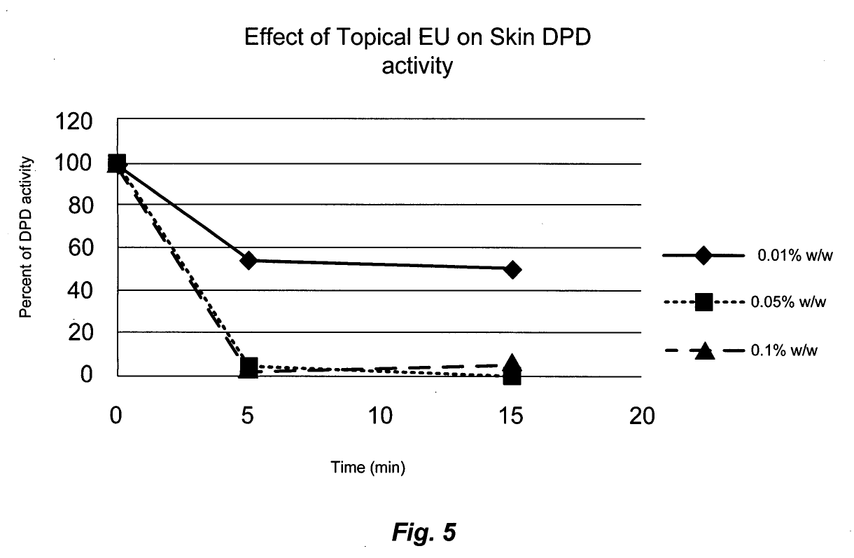

A baseline 5-FU pharmacokinetic study was conducted on Day-3 by collecting serial blood samples after 5-FU administration. On Day-2, pigs were either given 0.005% w/w eniluracil ointment (n=4 pigs), 0.01% w/w eniluracil ointment (n=4 pigs) or no ointment (n=2 pigs). A dose of 617 mg eniluracil ointment (0.01% and 0.005% w/w) was applied to a designated area that was 2.5 inches by 2.5 inches. The total ointment applied was 154.25 mg/m². The area of application in the pig (2.5 in²=0.0040 m²) was selected based on feasibility and scale to the proposed human use using the following figures: the surface area of one palm and one sole combined in a human is 2% of the total body surface area, the average total body surface area in a pig is 0.40 m². Thus, in terms of total eniluracil in mg/m² applied to pigs was 0.154 for 0.01% and 0.077 for 0.005%. The duration of application was 5 minutes, which was determined based data obtained from a pilot study. In this study, higher doses of topical eniluracil (5 gms of 0.01%, 0.05% and 0.01% w/w) and placebo were applied on the skin and skin biopsies were collected after predefined time intervals. These samples were then analyzed for DPD activity. Results of DPD activity Vs time is shown in FIG. 5, which demonstrates that only a short duration of exposure is needed to affect skin DPD activity.

Ointment exposure time was 5 minutes every other day for 16 days. Eniluracil treated animals also received 617 mg of placebo at a site different from the treatment site. Eniluracil pharmacokinetics were analyzed from the serial blood samples drawn on first day of the 5-FU and eniluracil treatment (Day 0) and on Day 14. In addition, eniluracil concentrations were evaluated in all samples obtained for 5-FU pharmacokinetics, as shown above. 5-FU pharmacokinetics were evaluated from 11 serial blood samples drawn after the IV bolus administration of 5-FU on Day-3, Day 0 and Day 14. On 5-FU treatment days, eniluracil administration was conducted first (5 min) followed by 5-FU at the 6 minute time point. Blood samples were collected for plasma EU analyses prior to topical administration and at 2 min and 5 min (end of topical exposure). Blood samples were collected for both EU and 5-FU analyses immediately prior to 5-FU administration and at the following times from the IV bolus: 2, 5, 10, 15, 30, 45, 60, 90 120, and 180 min. Determinations of eniluracil and 5-fluorouracil plasma pharmacokinetics were made. The effect on the local DPD activity and the effect of topical eniluracil on systemic DPD activity were assessed by collecting skin biopsies from both the treatment site and the placebo site, and peripheral blood mononuclear cells (PBMCs) on day-2 and on day 12. DPD activity in PBMCs was also measured on Day 17 and Day 19, to investigate the time to recovery if there was any systemic inhibition of DPD.

In terms of statistical considerations, the study was designed with 4 pigs to a treatment group in order to have 80% power for observation of at least a 40% difference in 5-FU systemic exposure between measurements conducted on day-3 (no eniluracil) to day 14 (on eniluracil every other day since day-2) at each of the two dosing levels (4 animals per dose level). These evaluations were conducted using matched, paired T Tests and Wilcoxon tests. In addition, day 14 data were compared between the treatment vs. control groups using the Kiruskal-Wallis test.

### Results & Discussion:

### Bioavailability of EU After Topical Administration:

Samples were drawn and evaluable in all animals studied for determination of plasma EU concentrations. A total of 224 samples were analyzed. No systemic eniluracil was observed above a limit of quantitation (11 ng/mL) for 221 of the 224 samples. Those 3 samples with measurable concentrations following EU (pig 5303 EU dose 0.005%: day 14, 6 min=12.81 ng/mL; day 14, 11 min=11.52 ng/mL; day 14, 15 min 11.07 ng/mL) were just above the assay lower limit of quantitation (11 ng/mL).

### Effect of Topical Eniluracil on Systemic 5-FU Levels:

To determine if application of topical eniluracil had any effect on 5-FU pharmacokinetics, animals were dosed with 5-FU on Day-3 (no topical eniluracil), Day 0 (2^{nd} day of topical eniluracil) and Day 14 (every other day topical eniluracil since day-2). Plasma samples were collected at predetermined time intervals, as described in the Methods section. Table 13 below summarizes the AUC and half-life values for each day.

TABLE 13
Mean (SD) Values for 5-FU Pharmacokinetic Data

| | Day −3 | Day 0 | Day 14 |
|---|---|---|---|
| AUC | | | |
| (mcg/mL * min) | | | |
| Dose 0.005% | 183.1 (65.00) | 277.0 (41.46) | 224.5 (144.5) |
| Dose 0.01% | 129.7 (26.54) | 182.6 (27.03) | 282.9 (112.0) |
| Both Doses | 156.4 (54.11) | 229.8 (59.98) | 253.7 (123.7) |
| Controls | 159.3 (40.26) | 182.7 (48.21) | 238.5 (112.6) |
| Half-Life (min) | | | |
| Dose 0.005% | 5.443 (1.710) | 6.976 (1.679) | 6.698 (1.581) |
| Dose 0.01% | 4.780 (1.773) | 13.19 (6.187) | 12.84 (8.570) |
| Both Doses | 5.111 (1.651) | 10.083 (5.352) | 9.771 (6.583) |
| Controls | 5.512 (0.556) | 7.556 (1.340) | 9.738 (4.825) |

Data from control animals were not statistically different compared to the treatment groups. In addition, intra-individual comparisons of 5-FU pharmacokinetics showed no observable influence of eniluracil administration on 5-FU plasma disposition (not shown). Furthermore, statistical analysis of the paired data showed no significant differences (P>0.05) in the 5-FU AUC or half-lives compared to prior to eniluracil administrations.

### Effect of Topical EU on PBMCs DPD Activity:

Animals (n=4 per group) were treated with topical eniluracil for 5 minutes on Day-2, then every other day until Day 14. Blood samples were collected on day-2, day 12, at time 0 (prior to application of topical ointment), 6 minutes (just after the removal of ointment) and at 60 minutes. There was no treatment on Day 17 and Day 19, but blood was collected on these days for PBMCs isolation to confirm that if there was any inhibition of DPD activity, it has recovered after the cessation of the treatment on Day 14. Tables 14 and 15 below show the mean DPD activity in the PBMC's after treatment with topical Eniluracil on Day-2, Day 12, Day 17, and Day 19.

TABLE 14
DPD activity (mean ± SE, n = 4) in the PBMCs of the pigs on
Day −2, 12, 17 and 19, this group was treated with 0.01% of Eniluracil
ointment (Starting at Day −2, then every other day until Day 14).

| | Day −2 | Day 12 | Day 17 | Day 19 |
|---|---|---|---|---|
| 0 Minutes | 155.67 ± 23.85 | 158.71 ± 24.30 | 201.73 ± | 127.09 ± |
| | | | 14.05 | 13.56 |
| 6 Minutes | 150.04 ± 42.29 | 172.43 ± 26.10 | 182.15 ± | 131.03 ± |
| | | | 7.94 | 16.14 |
| 60 Minutes | 164.73 ± 39.63 | 195.33 ± 13.11 | 178.65 ± | 148.95 ± |
| | | | 5.79 | 11.97 |

TABLE 15
DPD activity (mean ± SE, n = 4) in the PBMCs of the pigs on
Day −2, 12, 17 and 19, this group was treated with 0.005% of
Eniluracil ointment (Starting at day −2 until Day 14).

| | Day −2 | Day 12 | Day 17 | Day 19 |
|---|---|---|---|---|
| 0 Minutes | 142.57 ± 18.96 | 197.70 ± 30.12 | 146.25 ± | 161.43 ± |
| | | | 45.73 | 31.39 |
| 6 Minutes | 132.30 ± 26.05 | 216.19 ± 42.51 | 141.49 ± | 166.77 ± |
| | | | 73.98 | 44.25 |
| 60 Minutes | 131.11 ± 19.68 | 186.74 ± 30.45 | 181.61 ± | 197.60 ± |
| | | | 47.28 | 54.09 |

Statistical analysis showed no significant differences (P>0.05) in the DPD activity on Day-2, Day 12, Day 17, and Day 19, for both formulations.

### Effect of Topical EU on Skin DPD Activity:

Animals (n=4 per group) were treated with placebo and topical eniluracil for 5 minutes on Day-2, then every other day until Day 14^{th}. Skin samples were collected on day-2, day 12, at time 0 (prior to application of topical ointment), 6 minutes (just after the removal of ointment) and at 60 minutes. Samples were instantly frozen for subsequent analysis of DPD activity. Tables 16 and 17 below show the mean DPD activity in the skin after treatment with the two topical Eniluracil formulations on days-2 and 12.

TABLE 16
DPD activity (mean ± SE, n = 4) in the skin of the pigs on
Days −2, 12. This group was treated with 0.01% of eniluracil
ointment (Starting at day −2 every other day until day 14) and placebo.

| | Treatment | | Placebo | |
|---|---|---|---|---|
| | Day −2 | Day 12 | Day −2 | Day 12 |
| 0 Minutes | 11.78 ± 3.83 | 9.19 ± 7.08 | 6.06 ± 0.92 | 5.16 ± 3.52 |
| 5 Minutes | 4.72 ± 1.70 | 2.42 ± 2.04 | 4.20 ± 0.84 | 3.71 ± 2.63 |
| 60 Minutes | 8.60 ± 1.78 | 5.17 ± 4.01 | 8.51 ± 4.02 | 10.70 ± 5.88 |

TABLE 17
DPD activity (mean ± SE, n = 4) in the skin of the pigs on
Days −2, 12. This group was treated with 0.005% of eniluracil
ointment (Starting at day −2 every other day until day 14) and placebo.

| | Treatment | | Placebo | |
|---|---|---|---|---|
| | Day −2 | Day 12 | Day −2 | Day 12 |
| 0 Minutes | 14.92 ± 0.99 | 13.46 ± 7.02 | 8.43 ± 3.40 | 22.38 ± 13.01 |
| 5 Minutes | 8.32 ± 1.04 | 2.58 ± 1.00 | 5.26 ± 3.22 | 10.05 ± 5.19 |
| 60 Minutes | 8.12 ± 1.49 | 3.58 ± 1.35 | 8.51 ± 3.34 | 15.34 ± 2.54 |

These results demonstrate that short term topical application of eniluracil at 0.005% or 0.01% w/w resulted in temporary and recoverable inhibition of skin DPD activity, but did not significantly affect systemic 5-FU pharmacokinetics or systemic DPD activity.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Chazal M, Etienne M C, Renee N, et al. Link between dihydropyrimidine dehydrogenase activity in peripheral blood mononuclear cells and liver. Clin Cancer Res 1996;2:507-10.
Fischel J L, Formento P, Ciccolini J, et al. Lack of contribution of dihydrofluorouracil and alpha-fluoro-beta-alanine to the cytotoxicity of 5′-deoxy-5-fluorouridine on human keratinocytes. Anticancer Drugs. 2004; 15 :969-74.
Ferrero J M, Lassalle S, Mari M, et al. Hand-foot syndrome in patients receiving capecitabine: A pharmacological explanation. Proc Am Soc Clin Oncol 2006;24:2019.
Fourie J, Guarcello V, Diasio R B. Dose dependent inhibition of uridine phosphorylase (UP) by eniluracil (EU): Was the clinical inferiority of the EU/5-FU phase III trials due to an unrecognized inhibition of 5-FU anabolism? Abstract, 2006 ASCO Annual 2006
Grem J L, Harold N, Shapiri J, et al. Phase I and pharmacokinetic trial of weekly oral fluorouracil given with eniluracil and low dose leucovorin to patients with solid tumors. J Clin Oncol. 2000;18(23):3952-3963.
Guo X, Harold N, Saif M W, et al. Pharmacokinetic and pharmacodynamic effects of oral eniluracil, fluorouracil and leucovorin given on a weekly schedule. Cancer Chemother Pharmacol. 2003;52:79-85.
Keith B, Guo X, Zentko S, et al. Impact of two weekly schedules of oral eniluracil given with fluorouracil and leucovorin on the duration of dihydropyrimidine dehydrogenase inhibition. Clin Cancer Res. 2002;8:1045-1050.
Lu Z, Zhang R, Diasio R B. Dihydropyrimidine dehydrogenase activity in human peripheral blood mononuclear cells and liver: population characteristics, newly identified deficient patients, and clinical implications in 5-fluorouracil chemotherapy. Cancer Res 1993;53:5433.
Llovet J M, Ruff P, Tassopoulos N, et al. A phase II trial of oral eniluracil/5-fluorouracil in patients with inoperable hepatocellular carcinoma. Eur J Cancer 2001;37:1352-1358.
Rothenberg M L, Benedetti J K, Macdonald J S, et al. Phase II trial of 5-fluorouracil plus eniluracil in patients with advanced pancreatic cancer: a Southwest Oncology Group study. Ann Oncol. 2002; 13:1576-82.
Schilsky R L, Levin J, West W H, et al. Randomized, open-label, phase III study of a 28-day oral regimen of eniluracil plus fluorouracil versus intravenous fluorouracil plus leucovorin as first-line therapy in patients with metastatic/advanced colorectal cancer. J Clin Oncol. 2002;20(6): 1519-1526.
Smith I E, Johnston S R D, O'Brien M E R, et al. Low-dose oral fluorouracil with eniluracil as first-line chemotherapy against advanced breast cancer: A phase II study. J Clin Oncol 2000;18:2378-84.
Therasse P, Arbuck S G, Eisenhauer E A, et al. New guidelines to evaluate the response to treatment in solid tumors. European Organization for Research and Treatment of Cancer, National Cancer Institute of the United States, National Cancer Institute of Canada. J Natl Cancer Inst. 2000;92(3):205-216.

1. A method for reducing the frequency and/or severity of Hand-Foot Syndrome (HFS) in a patient undergoing treatment with 5-FU or a 5-FU prodrug, the method comprising applying to the hands and/or feet of said patient a topical formulation comprising an effective dose of an irreversible DPD inhibitor.

2. The method of claim 1, wherein the topical formulation inhibits DPD activity in the hands and/or feet but does not substantially inhibit systemic DPD activity in the patient.

3. The method of claim 1, wherein the concentration of DPD inhibitor in the topical formulation is about 0.001 to about 0.05 w/w.

4. The method of claim 1, further comprising the step of removing the topical formulation after an exposure time of about 5 to about 30 minutes.

5. The method of claim 1, wherein the topical formulation is in a form selected from the group consisting of an ointment, cream, lotion, aerosol spray, roll-on liquid and pad form.

6. The method of claim 1, wherein the topical formulation is applied prior to 5-FU or 5-FU prodrug treatment.

7. The method of claim 1, wherein the topical formulation is applied about 5 minutes to about 72 hours prior to 5-FU or 5-FU prodrug treatment.

8. The method of claim 1, wherein the DPD inhibitor comprises a 5-substituted uracil compound or a prodrug thereof.

9. The method of claim 1, wherein the DPD inhibitor comprises a uracil compound substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group, a C₁₋₄ alkyl group or a C₁₋₄ alkyl group substituted by halogen.

10. The method of claim 1, wherein the DPD inhibitor comprises a uracil compound selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil and 5-(2-bromo-1-chlorovinyl)uracil.

11. The method of claim 1, wherein the DPD inhibitor is selected from the group consisting of 5-(phenylselenenyl)uracil (PSU), 5-(phenylthio)uracil (PTU), 5-(phenylselenenyl)barbituric acid and 5-(phenylthio)barbituric acid.

12. The method of claim 1, wherein the DPD inhibitor is also an inhibitor of TP and/or UP or the topical formulation further comprises a TP and/or UP inhibitor.

13. The method of claim 1, wherein the DPD inhibitor is eniluracil or a prodrug thereof.

14. The method of claim 1, wherein the 5-FU or 5-FU prodrug is selected from the group and their 5′-esters, including phosphate esters: consisting of 5-fluorouridine, 5-fluorocytidine, 5-fluoro-2-deoxyuridine, 5-fluoro-2-deoxycytidine, 5′-deoxy-4′5-fluorouridine, and 5-fluoroarabinosyluracil. 5′-Deoxy-5-fluorouridine, 1-(2-tetrahydrofuranyl)-5-fluorouracil, 1-C₁₋₈ alkylcarbamoyl-5-fluorouracil derivative, 1-(2-tetrahydrofuryl)-5-fluorouracil, 5′-deoxy-5-fluoro-N-[(pentyloxy)carbonyl]-cytidine (capecitabine), or a compound that is converted to 5-FU in vivo.

15. The method of claim 1, wherein the 5-FU or 5-FU prodrug is 5-FU or capecitabine.

16. The method of claim 1, wherein the DPD inhibitor is eniluracil and the 5-FU or 5-FU prodrug is 5-FU or capecitabine.

17. A topical formulation for reducing the frequency and/or severity of Hand-Foot Syndrome (HFS) in a patient undergoing treatment with 5-FU or a 5-FU prodrug, the topical formulation comprising an effective dose of an irreversible DPD inhibitor.

18. The topical formulation of claim 17, wherein the effective dose inhibits DPD activity in the hands and/or feet but does not result in substantial systemic DPD inhibition.

19. The topical formulation of claim 17, wherein the concentration of DPD inhibitor in the topical formulation is about 0.001 to about 0.05 w/w.

20. The topical formulation of claim 17, wherein the DPD inhibitor comprises a 5-substituted uracil compound or a prodrug thereof.

21. The topical formulation of claim 17, wherein the DPD inhibitor comprises a uracil compound substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group, a C₁₋₄ alkyl group or a C₁₋₄ alkyl group substituted by halogen.

22. The topical formulation of claim 17, wherein the DPD inhibitor comprises a uracil compound selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil and 5-(2-bromo-1-chlorovinyl)uracil.

23. The topical formulation of claim 17, wherein the DPD inhibitor is selected from the group consisting of 5-(phenylselenenyl)uracil (PSU), 5-(phenylthio)uracil (PTU), 5-(phenylselenenyl)barbituric acid and 5-(phenylthio)barbituric acid.

24. The topical formulation of claim 17, wherein the DPD inhibitor is also an inhibitor of TP and/or UP or the topical formulation further comprises a TP and/or UP inhibitor.

25. The topical formulation of claim 17, wherein the DPD inhibitor is eniluracil or a prodrug thereof.

26. The topical formulation of claim 17, wherein the topical formulation is selected from the group consisting of an ointment, cream, lotion, aerosol spray, roll-on liquid and pad form.
